(19) United States
(12) Patent Application Publication
(10) Pub. No.: US 2020/0008879 A1
(43) Pub. Date: Jan. 9, 2020

(54) ULTRASOUND GUIDANCE OF ACTUATABLE MEDICAL TOOL

(71) Applicant: KONINKLIJKE PHILIPS N.V., EINDHOVEN (NL)

(72) Inventors: Aleksandra POPOVIC, BOSTON, MA (US); Ralf SEIP, CARMEL, NY (US)

(21) Appl. No.: 16/470,266

(22) PCT Filed: Dec. 14, 2017
(86) PCT No.: PCT/EP2017/082922

§ 371 Date: Jun. 17, 2019

Related U.S. Application Data
(60) Provisional application No. 62/435,945, filed on Dec. 19, 2016.

(51) Int. Cl. A61B 34/20; A61B 8/08; A61B 8/00

(52) U.S. Cl. CPC A61B 34/20; A61B 8/0841; A61B 2034/2063; A61B 8/54; A61B 8/5207

(57) ABSTRACT

An ultrasound sensing guidance system employing a medical tool (30) including an ultrasonic motor (40) for actuating the medical tool (30) relative to an anatomical region. The ultrasound sensing guidance system further employs an ultrasound transducer (50) and an ultrasound sensing guidance controller (70). In operation, the ultrasound transducer (50) generates acoustic sensing data indicative of a sensing by the ultrasound transducer (50) of an acoustic wave emitted by the ultrasonic motor (40) as the ultrasonic motor (40) actuates the medical tool (30) relative to the anatomical region, and the ultrasound sensing guidance controller (70) controls an actuation of the medical tool (30) by the ultrasonic motor (40) responsive to the generation of the acoustic sensing data by the ultrasound transducer (50).

### FIELD OF THE INVENTION

The inventions of the present disclosure generally relate to ultrasound guidance systems (e.g., the Sparq Ultrasound System, the Epiq Ultrasound System, the SonixGPS Ultrasound Guidance System, the ACUSON S3000™ Ultrasound System, the flex Focus 400exp Ultrasound System, etc.). The inventions of the present disclosure more particularly relate to improving such ultrasound guidance systems by providing an ultrasound guidance of a medical tool actuatable by an ultrasonic motor.

### BACKGROUND OF THE INVENTION

Actuation of medical tools (e.g., interventional and surgical tools/instruments) inside a patient body is required for many interventional and surgical tasks: tool alignment, punctures, drilling, etc. Today, actuation is primarily used in open surgery due to size of the actuation mechanisms. This size is guided by the dimensions of the actuation motors. Traditional electric motors consist of many parts (e.g. permanent magnets, coils, commutators, etc.), which limit the possibilities for miniaturization. In addition, these motors cause electromagnetic disturbances which may interfere with therapy devices and medical imaging devices.

An alternative to electromagnetic motors are piezo-electric based ultrasonic motors. The actuation is achieved by applying a time-varying voltage to the piezo-electric crystals making up the ultrasonic motor, causing expansion and contraction of the material. This mechanical oscillation may be converted into rotational motion or linear motion mimicking traditional electromagnetic motors. Some of the advantages of piezo-electric based ultrasonic motors are: scalability (they can be made very small), unlimited linear motion (not lead-screw dependent), high power per volume and no EM disturbance.

While advantageous, a disadvantage of piezo-electric based ultrasonic motors is that they require additional sensors to measure the amount and speed of displacement of the actuatable component (e.g., a shaft, a disk, etc.). This leads to inaccurate or open-loop position control, which may be a limiting factor for medical applications. The inaccuracies are coming from several main sources.

One source is an inaccurate physical model of a piezo-electric based ultrasonic motor. Specifically, although control models of piezo-electric based ultrasonic motors are known in the art, they are non-linear and dependent on the environment (e.g. temperature, requested torque/load) and thus the prior control models of piezo-electric based ultrasonic motors have proven unreliable.

A second source is an interaction of piezo-electric based ultrasonic motors with the environment. More particularly, a piezo-electric based ultrasonic motor may experiences resistance (e.g. from tissue) that may not be detected and compensated for by an open-loop control system.

Finally, delays in the system caused by time or inertia to translate motion signals to oscillation of the piezo-electric crystals that will cause the motion.

Some methods to alleviate these problems include the use of displacement sensors such as Hall-effect sensors, resistive sensors, or optical encoders. These sensors have to be mounted close to or on to the piezo-electric based ultrasonic motor itself, increasing the size of the mechanism. Also, such sensors may not have sufficient resolution to detect interaction by the piezo-electric based ultrasonic motor with the environment.

### SUMMARY OF THE INVENTION

To improve upon ultrasound guidance systems, the present disclosure provides inventions for controlling a medical tool having an ultrasonic motor by using a combined ultrasound imaging of the medical tool and acoustic sensing of the ultrasonic motor to thereby accurately position the medical tool within an anatomical region.

One embodiment of the inventions of the present disclosure is an ultrasound sensing guidance system employing a medical tool including an ultrasonic motor for actuating the medical tool relative to anatomical region.

The ultrasound sensing guidance system further employs an ultrasound transducer and an ultrasound sensing guidance controller.

In operation, the ultrasound transducer generates acoustic sensing data indicative of a sensing by the ultrasound transducer of an acoustic wave emitted by the ultrasonic motor as the ultrasonic motor actuates the medical tool relative to the anatomical region, and the ultrasound sensing guidance controller controls an actuation of the medical tool by the ultrasonic motor responsive to the generation of the acoustic sensing data by the ultrasound transducer.

A second embodiment of the inventions of the present disclosure is the ultrasound sensing guidance controller employing a tool actuation detector and an ultrasonic motor actuator.

In operation, the tool actuation detector detects an actuation of the medical tool by the ultrasonic motor within an anatomical region responsive to a generation by the ultrasound transducer of acoustic sensing data indicative of a sensing by the ultrasound transducer of an emission of an acoustic wave by the ultrasonic motor as the ultrasonic motor actuates the medical tool within the anatomical region.

The medical tool actuator controls the actuation of the medical tool by the ultrasonic motor within the anatomical region responsive to a detection by the tool actuation detector of the actuation of the medical tool by the ultrasonic motor within the anatomical region.

A third embodiment of the inventions of the present disclosure is an ultrasound sensing guidance method.

The ultrasound sensing guidance method involves an ultrasound sensing guidance controller controlling an actuation of the medical tool by the ultrasonic motor within an anatomical region.

The ultrasound sensing guidance method further involves the ultrasound transducer generating acoustic sensing data indicative of a sensing by the ultrasound transducer of an emission of an acoustic wave by the ultrasonic motor as the ultrasonic motor actuates the medical tool within the anatomical region, wherein the ultrasound sensing guidance controller controls the actuation of the medical tool by the ultrasonic motor within the anatomical region responsive to the generation of the acoustic sensing data by the ultrasound transducer.

For purposes of describing and claiming the inventions of the present disclosure:

(1) the term “ultrasound sensing guidance system” broadly encompasses all ultrasound guidance systems, as known in the art of the present disclosure and hereinafter conceived, incorporating the inventive principles of the present disclosure for controlling a medical tool having an ultrasonic motor by using a combined ultrasound imaging of the medical tool and acoustic sensing of the ultrasonic motor to thereby accurately position the medical tool within an anatomical region. Examples of known ultrasound guidance systems include, but are not limited to, the Sparq Ultrasound System, the Epiq Ultrasound System, the SonixGPS Ultrasound Guidance System, the ACUSON S3000™ Ultrasound System, the flex Focus 400exp Ultrasound System;

(2) the term “ultrasound sensing guidance method” broadly encompasses all ultrasound guidance methods, as known in the art of the present disclosure and hereinafter conceived, incorporating the inventive principles of the present disclosure for controlling a medical tool having an ultrasonic motor by using a combined ultrasound imaging of the medical tool and acoustic sensing of the ultrasonic motor to thereby accurately position the medical tool within an anatomical region;

(3) the term “medical tool” broadly encompasses any and all types of medical tools, as known in the art of the present disclosure and hereinafter conceived, for performing one or more specific tasks in support of any type of medical procedure including, but not limited to, diagnostic, therapeutic and surgical procedures;

(4) the term “actuation” or any tense thereof broadly encompasses a mechanical motion in the form of a translation, a rotation/or a pivoting;

(5) the term “ultrasonic motor” broadly encompasses all electronic motors, as known in the art of the present disclosure and hereinafter conceived, powered by an ultrasonic vibration of component(s) including, but not limited to, a piezo-electric component(s);

(6) the term “ultrasound transducer” broadly encompasses any and all ultrasound transducers, as known in the art of the present disclosure and hereinafter conceived, suitable for generating for emitting and receiving ultrasound waves. Examples of an ultrasound transducer include, but are not limited to, a Transesophageal echocardiography (TEE) probe, an Intra-cardiac probe (ICE), intra-nasal probe and a intravascular ultrasound (IVUS) probe;

(7) the term “controller” broadly encompasses all structural configurations of an application specific main board or an application specific integrated circuit housed employed within or linked to an ultrasound sensing guidance system of the present disclosure for controlling an application of various inventive principles of the present disclosure as subsequently exemplarily described herein. The structural configuration of the controller may include, but is not limited to, processor(s), computer-usable/computer readable storage medium(s), an operating system, application module(s), peripheral device controller(s), interface(s), bus(es), slot(s) and port(s);

(8) the term “application module” broadly encompasses a component of a robot controller consisting of an electronic circuit and/or an executable program (e.g., executable software and/or firmware stored on non-transitory computer readable medium(s)) for executing a specific application; and

(9) the terms “signal”, “data”, and “command” broadly encompasses all forms of a detectable physical quantity or impulse (e.g., voltage, current, or magnetic field strength) as understood in the art of the present disclosure and as exemplary described herein for communicating information and/or instructions in support of applying various inventive principles of the present disclosure as subsequently described herein. Signal/data/command communication between components of the present disclosure may involve any communication method, as known in the art of the present disclosure and hereinafter conceived, including, but not limited to, signal/data/command transmission/reception over any type of wired or wireless medium/datalink and a reading of signal/data/command uploaded to a computer-usable/computer readable storage medium.

The foregoing embodiments and other embodiments of the inventions of the present disclosure as well as various features and advantages of the inventions of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of the present disclosure being defined by the appended claims and equivalents thereof.

### BRIEF DESCRIPTION OF THE DRAWINGS

FIG. 1 illustrates an exemplary embodiment of an ultrasound sensing guidance system in accordance with the inventive principles of the present disclosure.

FIG. 2A illustrates an exemplary embodiment of an ultrasound sensing of a medical device including a linear ultrasonic motor in accordance with the inventive principles of the present disclosure.

FIG. 2B illustrates an exemplary control loop of ultrasound sensing of a medical device including a linear ultrasonic motor of FIG. 2A in accordance with the inventive principles of the present disclosure.

FIG. 3A illustrates an exemplary embodiment of an ultrasound sensing of a medical device including a rotary ultrasonic motor in accordance with the inventive principles of the present disclosure.

FIG. 3B illustrates an exemplary control loop of ultrasound sensing of a medical device including a rotary ultrasonic motor of FIG. 3A in accordance with the inventive principles of the present disclosure.

FIG. 4 illustrates an exemplary embodiment of a steerable introducer as known in the art.

FIG. 5 illustrates an exemplary embodiment of the ultrasound sensing guidance system of FIG. 1 in accordance with the inventive principles of the present disclosure

FIG. 6 illustrates an exemplary registration of a steerable introducer and a transesophageal echocardiogram (TEE) probe in accordance with the inventive principles of the present disclosure.

FIG. 7 illustrates an exemplary embodiment of a flowchart representative of an ultrasound sensing guidance method in accordance with the inventive principles of the present disclosure.

FIG. 8 illustrates an exemplary control loop of the registered steerable introducer and TEE probe of FIG. 7 in accordance with the inventive principles of the present disclosure.

### DETAILED DESCRIPTION OF THE PREFERRED EMBODIMENTS

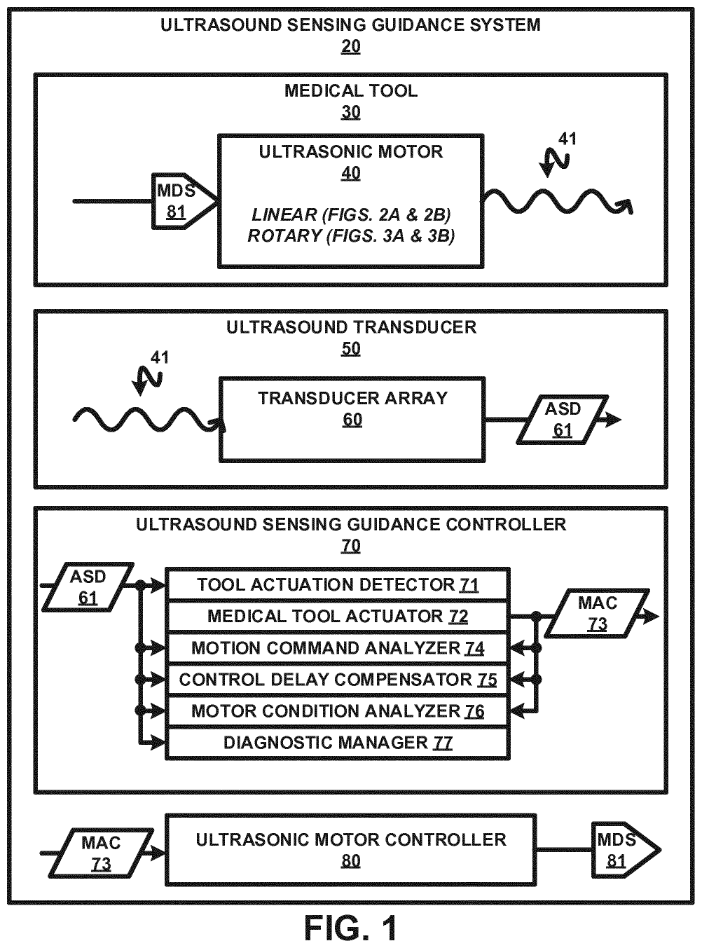

To facilitate an understanding of the inventions of the present disclosure, the following description of FIG. 1 teaches basic inventive principles of an ultrasound sensing guidance system in accordance with the inventive principles of the present disclosure. From this description of FIG. 1, those having ordinary skill in the art will appreciate how to apply the inventive principles of the present disclosure to practice numerous and various embodiments an ultrasound sensing guidance system in accordance with the inventive principles of the present disclosure.

In practice, the inventions of the present disclosure are applicable to any anatomical region including a cephalic region, a cervical region, a thoracic region, an abdominal region, a pelvic region, a lower extremity and an upper extremity.

Also in practice, the inventions of the present disclosure are applicable to any type of anatomical structure including but not limited to, tissue and bone, healthy or unhealthy.

Further in practice, the inventions of the present disclosure are applicable to any type of medical procedure, particularly interventional and surgical procedures.

Referring to FIG. 1, an ultrasound sensing guidance system 20 of the present disclosure employs a medical tool 30, an ultrasound transducer 50, an ultrasound sensing guidance controller 70 and an ultrasonic motor controller 80.

Medical tool 30 is any type of medical tool for performing a specific task during a medical procedure (e.g., an interventional tool, a surgical tool and a therapy tool). Medical tool 30 includes an ultrasonic motor 40 of any type for actuating medical tool 30 (e.g., an actuation of a translational motion, a rotary motion and a pivoting motion of medical tool 30). In practice, a time-varying motor drive signal 81 is applied to ultrasonic motor 40 to control the actuation of medical tool 30. As known in the art of the present disclosure, ultrasonic motor 40 emits an acoustic wave 41 as time-varying motor drive signal 81 is applied to ultrasonic motor 40.

Ultrasound transducer 50 includes a transducer array 60 of any type for converting electrical signal(s) to ultrasound wave(s) and converting ultrasound wave(s) to electrical signal(s). In practice, an anatomical region imaging by ultrasound transducer 50 as known in the art of the present disclosure involves an emission and a reception of ultrasound waves by transducer array 60 for imaging an anatomical region (not shown in FIG. 1). Further in practice, as shown in FIG. 1, a sensing of ultrasonic motor 40 by ultrasound transducer 50 in accordance with the inventive principles of the present disclosure involves a reception by transducer array 60 of acoustic wave 41 emitted by ultrasound motor 40 during an actuation of medical tool 30 and a generation by transducer array 60 of acoustic sensing data 61 informative of waveform characteristics of acoustic wave 41 (e.g., frequency, amplitude, pulse repetition, etc.) distinctive from waveform characteristics of the ultrasound waves emitted and received by transducer array 60 for imaging an anatomical region.

In one embodiment, a subset of elements of transducer array 60 are exclusively designated for anatomical region imaging and another subset of elements of transducer array 60 are exclusively designated for ultrasonic motor sensing.

In another embodiment, an entirety of transducer array 60 may be alternated between the anatomical region imaging and the ultrasonic motor sensing.

In yet another embodiment, an entirety of transducer array 60 may designed for ultrasonic motor sensing while a second ultrasound transducer or other imaging modality may be utilized for the anatomical region imaging.

Ultrasound sensing guidance controller 70 controls an actuation of medical tool 30 by ultrasonic motor 40 in accordance with the inventive principles of the present disclosure. In practice, ultrasound sensing guidance controller 70 may be structurally configured to execute application module(s) for controlling an actuation of medical tool 30 by ultrasonic motor 40 including, but not limited to, a tool actuation detector 71, a medical tool actuator 72, a motion command analyzer 74, a control delay compensator 75, a motion analyzer 76 and a diagnostic manager 77.

Tool actuation detector 71 implements a detection of an actuation of medical tool 30 achieved by ultrasonic motor 40 relative to transducer array 60 as will be further exemplary described herein. In practice, position of peaks of acoustic wave 41 as indicated by acoustic sensing data 61 may be utilized as known in the art of the present disclosure to detect an exact actuation position of ultrasonic motor 40 relative to transducer array 60.

Medical tool actuator 72 implements a generation of a motor actuation command 73 for actuating medical tool from the actuation position detected by tool actuation detector 71 to a target actuation position as will be further exemplary explained herein. Motor actuation command 73 is processed by an ultrasonic motor controller 80 via a kinematic model of ultrasonic motor 40 to generate time-varying motor drive signal 81 as known in the art of the present disclosure.

Motion command analyzer 74 implements an analysis of the waveform of acoustic wave 41 to ascertain a status of a control loop between an issuance of motor actuation command 73 by medical tool actuator 72 and a receipt and/or an execution of motor drive signal 81 by ultrasonic motor 40 as known in the art of the present disclosure whereby medical tool actuator 72 may adjust the control loop as necessary during a generation of motor actuation command 73.

Control delay compensator 75 implements a detection of a time duration of a peak of the acoustic wave 41 to thereby communicate a time delay to medical tool actuator 72 whereby medical tool actuator 72 may implement a delay compensation scheme as known in art of the present disclosure during a generation of motor actuation command 73.

Motion analyzer 76 implements a determination of an actuation condition of ultrasonic motor 40 by analysis of any differences between an expected waveform of acoustic wave 41 in accordance with motor actuation command 73 and an actual received waveform of acoustic wave 41, particularly in term of an amplitude and a spectral content of acoustic wave 41. Any significant difference between the expected waveform and actual received waveform of acoustic wave 41 may be indicative of an actuation condition of ultrasonic motor 40 including, but not limited to, an overloading of ultrasonic motor 40 or a deteriorating operation of ultrasonic motor 40. Such conditions would occur when medical tool 30 is being actuated to a target actuation position where medical tool 30 encounters resistance, which in turn manifests itself as a change in emanated acoustic wave 41 from ultrasonic motor 40. In such cases, motor control command 73 may not control an actual motor displacement (e.g., due to boundary conditions on the piezoelectric crystal), resulting in a missing ultrasound signature from acoustic wave 41 where there should have been one. This determination by motion analyzer 76 may be communicated via a delineation on the anatomical region imaging to facilitate an operator controlled adjustment as necessary in the positioning of medical tool 30.

Based on any determination of an actuation condition by motion analyzer 76, ultrasound sensing guidance controller 70 may perform any number of remedies and/or user notifications including, but not limited to, activating an alarm condition, shutdown ultrasound sensing guidance system 20 and calling a remedy algorithm to address the actuation condition.

Diagnostic manager 77 implements a remote diagnosis of any problem of ultrasonic motor 77 based on waveform characteristics of acoustic wave 30, particularly an amplitude and/or a spectral content of acoustic wave 30. An example of potential problems include a broken piezo element/connection of ultrasonic motor 40 indicated by an absence of an expected emanation of acoustic wave 41 by ultrasonic motor 40.

In practice, one structural embodiment of ultrasound sensing guidance controller 70 may include a processor, a memory, a user interface, a network interface, and a storage interconnected via one or more system buses.

The processor may be any hardware device, as known in the art of the present disclosure or hereinafter conceived, capable of executing instructions stored in memory or storage or otherwise processing data. In a non-limiting example, the processor may include a microprocessor, field programmable gate array (FPGA), application-specific integrated circuit (ASIC), or other similar devices.

The memory may include various memories, as known in the art of the present disclosure or hereinafter conceived, including, but not limited to, L1, L2, or L3 cache or system memory. In a non-limiting example, the memory may include static random access memory (SRAM), dynamic RAM (DRAM), flash memory, read only memory (ROM), or other similar memory devices.

The user interface may include one or more devices, as known in the art of the present disclosure or hereinafter conceived, for enabling communication with a user such as an administrator. In a non-limiting example, the user interface may include a display, a mouse, and a keyboard for receiving user commands. In some embodiments, the user interface may include a command line interface or graphical user interface that may be presented to a remote terminal via the network interface.

The network interface may include one or more devices, as known in the art of the present disclosure or hereinafter conceived, for enabling communication with other hardware devices. In an non-limiting example, the network interface may include a network interface card (NIC) configured to communicate according to the Ethernet protocol. Additionally, the network interface may implement a TCP/IP stack for communication according to the TCP/IP protocols. Various alternative or additional hardware or configurations for the network interface will be apparent\

The storage may include one or more machine-readable storage media, as known in the art of the present disclosure or hereinafter conceived, including, but not limited to, read-only memory (ROM), random-access memory (RAM), magnetic disk storage media, optical storage media, flash-memory devices, or similar storage media. In various non-limiting embodiments, the storage may store instructions for execution by the processor or data upon with the processor may operate. For example, the storage may store a base operating system for controlling various basic operations of the hardware. The storage may further store one or more application module(s) 71-77 in the form of executable software/firmware.

Also in practice, controllers 70 and 80 may be segregated, or may be partially or wholly integrated.

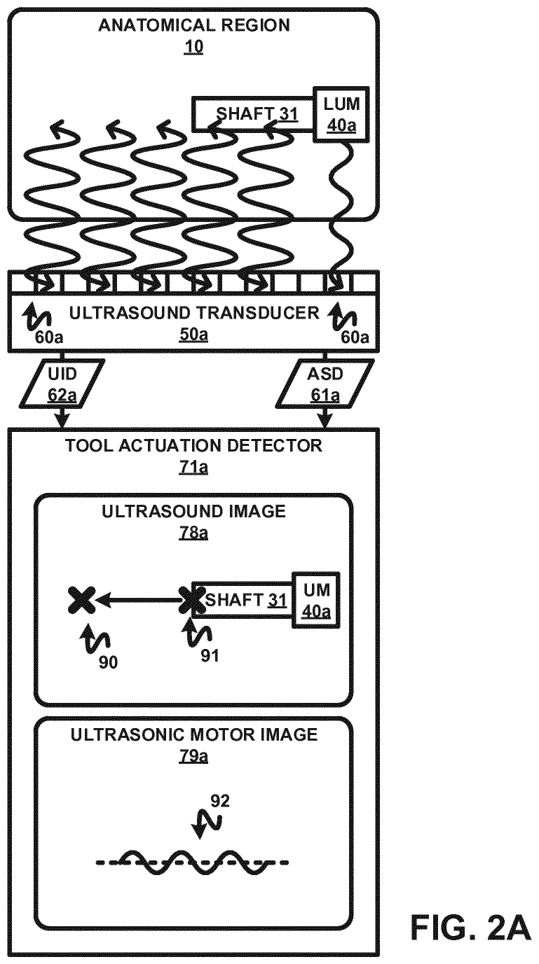

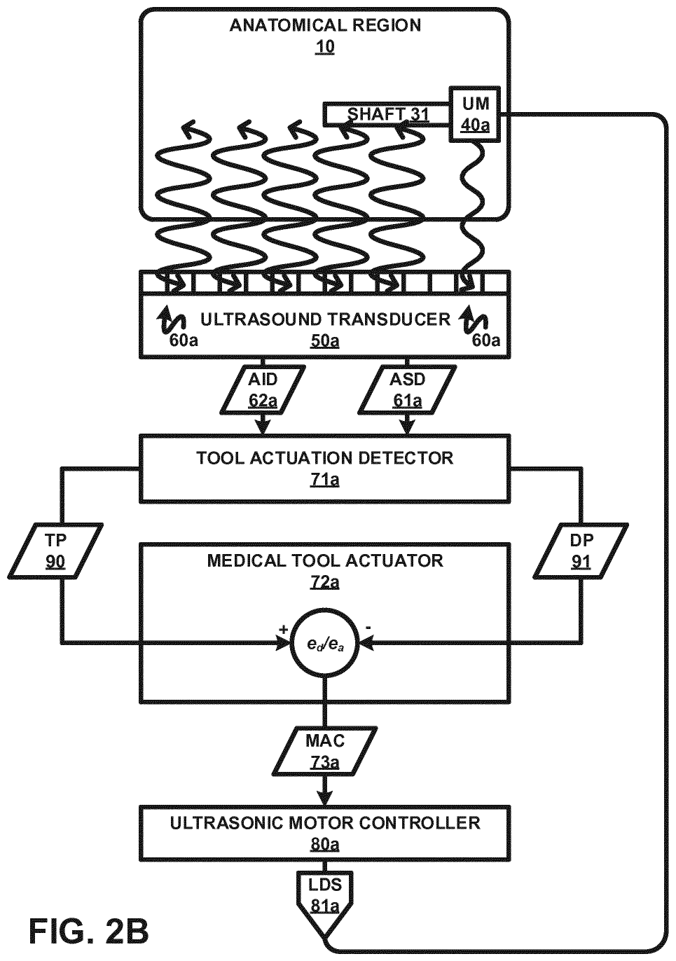

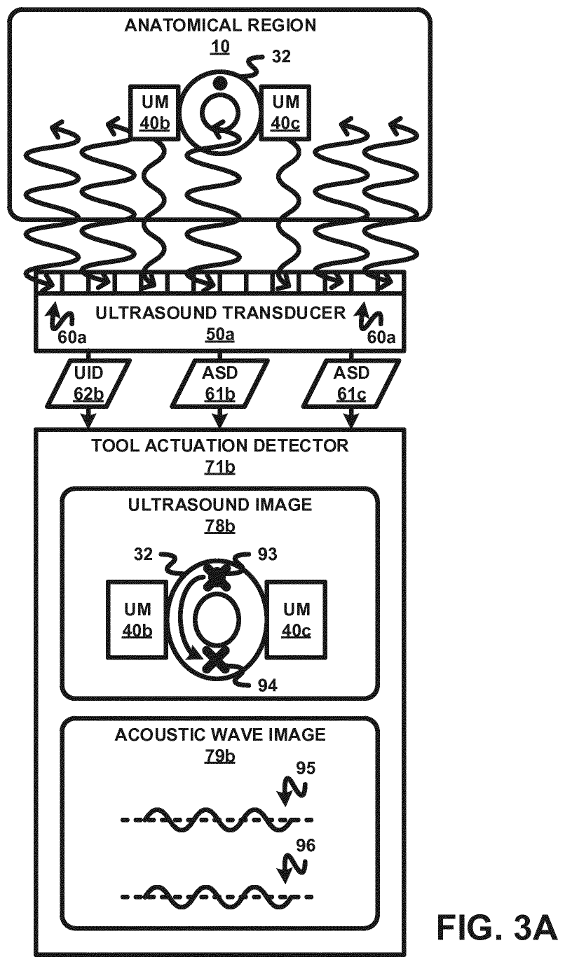

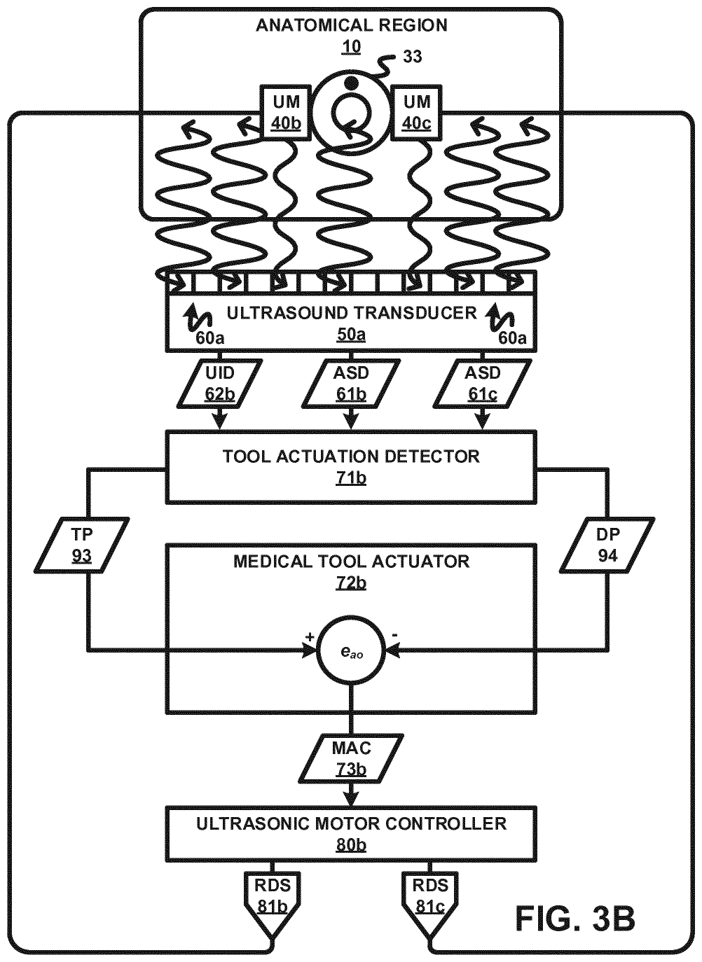

To facilitate a further understanding of the inventions of the present disclosure, the following description of FIGS. 2A-3B teaches basic inventive principles of an ultrasound sensing guidance method in accordance with the inventive principles of the present disclosure. From this description of FIGS. 2A-3B, those having ordinary skill in the art will appreciate how to apply the inventive principles of the present disclosure to practice numerous and various embodiments of ultrasound sensing guidance method in accordance with the inventive principles of the present disclosure.

Generally in practice, an ultrasound sensing guidance method of the present disclosure is based on:
1. an initial actuation of a medical tool 30 (FIG. 1) by ultrasound sensing guidance controller 70 (FIG. 1) toward a target actuation position within or adjacent to an anatomical region based on an initial detected actuation position of medical tool 30 within an image illustrative of medical tool 30 within or adjacent to an anatomical region (e.g., an ultrasound image generated by ultrasound transducer 50 (FIG. 1));
2. a sensing by ultrasound transducer 50 of acoustic wave 41 (FIG. 1) emitted by ultrasonic motor 40 (FIG. 1); and
3. further actuation of medical tool 30 by ultrasound sensing guidance controller 70 (FIG. 1) toward the target actuation position within or adjacent to the anatomical region based the sensing by ultrasound transducer 50 of acoustic wave 41 (FIG. 1).

In one linear ultrasonic embodiment, FIGS. 2A and 2B illustrate a control loop of an ultrasonic sensing guidance method of the present disclosure involving a linear ultrasonic motor 40a for linearly translation a shaft 31 representative of a medical tool 30 (FIG. 1) within an anatomical region 10.

Referring to FIGS. 2A and 2B, the control loop includes an ultrasound transducer 50a generating ultrasound image data 62a by an emission and a reception of ultrasound waves symbolized by the bi-directional waves within anatomical region 10 whereby an ultrasound image 78a illustrative of a positioning of shaft 31 within anatomical region 10 is utilized to delineate a target actuation position 90 of a distal end of shaft 31 within anatomical region 10 and to detect a current actuation position 91 of a distal end shaft 31 within anatomical region 10.

As shown in FIG. 2B, the control loop further includes a medical tool actuator 72a of an ultrasound sensing guidance controller of the present disclosure ascertaining a position distance error signal e_{d} as a dot differential between the center cᵥ of target actuation position 90 and the center c_{d} of detected actuation position 91 in accordance with the following equation [1]:

e_{d}=∥cᵥ−c_{d}∥ [1]

Medical tool actuator 72a may further ascertain an alignment error signal eₐ as a dot product of two planes normal indicative of an angular error in accordance with the following equation [2]:

eₐ=\vec{n}ᵥ·\vec{n}_{d} [2]

Medical tool actuator 72a may further ascertain a safety error signal eₛ (not shown) as a dot differential between the center cᵥ of target actuation position 90 and a spot location cₛ of an anatomical structure within anatomical region 10 closest detected actuation position 91 in accordance with the following equation [3]:

eₛ=|cₛ−c_{d}∥ [3]

From the position distance error signal and alignment error signal (if applicable), medical tool actuator 72a generates motion actuation command 73a for driving the error signal(s) to zero within the context of safety error signal (if applicable). Medical tool actuator 72a communicates motion actuation command 73a whereby ultrasound motor controller 80a processes the applicable error signal(s) as inputs to an inverse kinematics computation of shaft 31 to ascertain a linear drive signal 81a for ultrasonic motor 40a to thereby actuate the necessary translation of shaft 31 to target position 90.

As ultrasound motor 40a translates shaft 31 to target position 90, ultrasound transducer 50a generates acoustic sensing data 61a informative of a sensing of an acoustic wave emitted ultrasonic motor 40a whereby a motion actuation detector 71a of the ultrasound sensing guidance controller generates an acoustic wave image 79a of a waveform 92 of the acoustic wave as shown in FIG. 2A. By detecting the peaks of waveform 92 of the acoustic wave, motor actuation detector 71a ascertains the detected actuation position 91 of the distal end of shaft 31 whereby medical tool actuator 72a continually generates the error signal(s) until the distal end of shaft 31 reaches target actuation position 90.

In practice, the detected actuation position 91 may be detected based on both ultrasound image 78a and acoustic wave image 79a whereby one of the images serves as the primary source of detected actuation position 91 and the other image serves to confirm detected actuation position 91.

Also in practice, application modules 74-77 (FIG. 1) or equivalents thereof may be incorporated in support of the control loop.

In one rotary ultrasonic embodiment, FIGS. 3A and 3B illustrate a control loop of an ultrasonic sensing guidance method of the present disclosure involving a pair of rotary ultrasonic motors 40b and 40c for a rotation actuation of a disk 32 representative of a medical tool 30 (FIG. 1) within an anatomical region 10.

Referring to FIGS. 3A and 3B, the control loop includes ultrasound transducer 50a generating ultrasound image data 63B by an emission and a reception of ultrasound waves symbolized by the bi-directional waves within anatomical region 10 whereby an ultrasound image 78b illustrative of a positioning of disk 32 within anatomical region 10 is utilized to delineate a target actuation position 94 of a reference point of disk 32 within anatomical region 10 and to detect a current actuation position 93 of a reference point disk 32 within anatomical region 10.

As shown in FIG. 3B, the control loop further includes a medical tool actuator 72b of an ultrasound sensing guidance controller of the present disclosure ascertaining an angular orientation error signal eₐₒ as a dot differential between angular orientation aₛ of target actuation position 94 relative to a reference actuation position and an angular orientation a_{d} of detected actuation position 93 relative to a reference actuation position in accordance with the following equation [4]:

eₐₒ=∥aₛ−a_{d}∥ [4]

From the angular orientation error signal, medical tool actuator 72b generates motion actuation command 72b for driving the error signal to zero. Medical tool actuator 72b communicates motion actuation command 72b whereby ultrasound motor controller 80b processes the applicable error signal as inputs to an inverse kinematics computation of disk 32 to ascertain a rotary drive signals 81b and 81c respective for ultrasonic motor 40b and ultrasonic motor 40c to thereby actuate the necessary rotation of disk 32 to target position 93.

As ultrasound motor 40b and ultrasonic motor 40c rotates disk 32 to target position 93, ultrasound transducer 50a generates acoustic sensing data 61a and ultras sensing data 61b informative of a sensing of an acoustic wave emitted ultrasonic motor 40b whereby a motion actuation detector 71a of the ultrasound sensing guidance controller of the present disclosure generates an acoustic wave image 79ba of a waveform 95 of the acoustic wave emitted by ultrasonic motor 40b and of a waveform 9b of the acoustic wave emitted by ultrasonic motor 40c as shown in FIG. 3A. By detecting the peaks of waveform 95 and waveform 96 of the acoustic waves, motor actuation detector 71b ascertains the detected actuation position 9e of the reference point of disk 32 whereby medical tool actuator 72b continually generates the error signal(s) until the reference point of disk 32 reaches target actuation position 94.

In practice, the detected actuation position 93 may be detected based on both ultrasound image 78b and acoustic wave image 79a whereby one of the images serves as the primary source of detected actuation position 93 and the other image serves to confirm detected actuation position 93.

Also in practice, application modules 74-77 (FIG. 1) or equivalents thereof may be incorporated in support of the control loop.

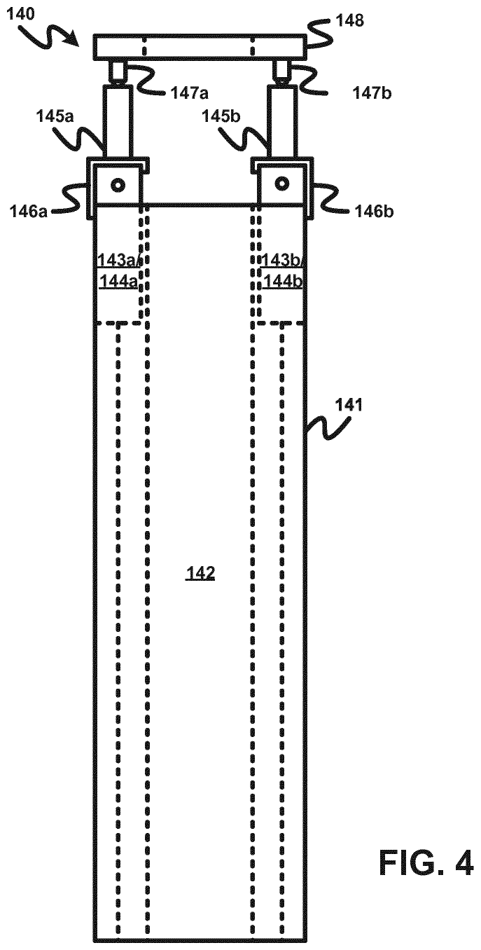

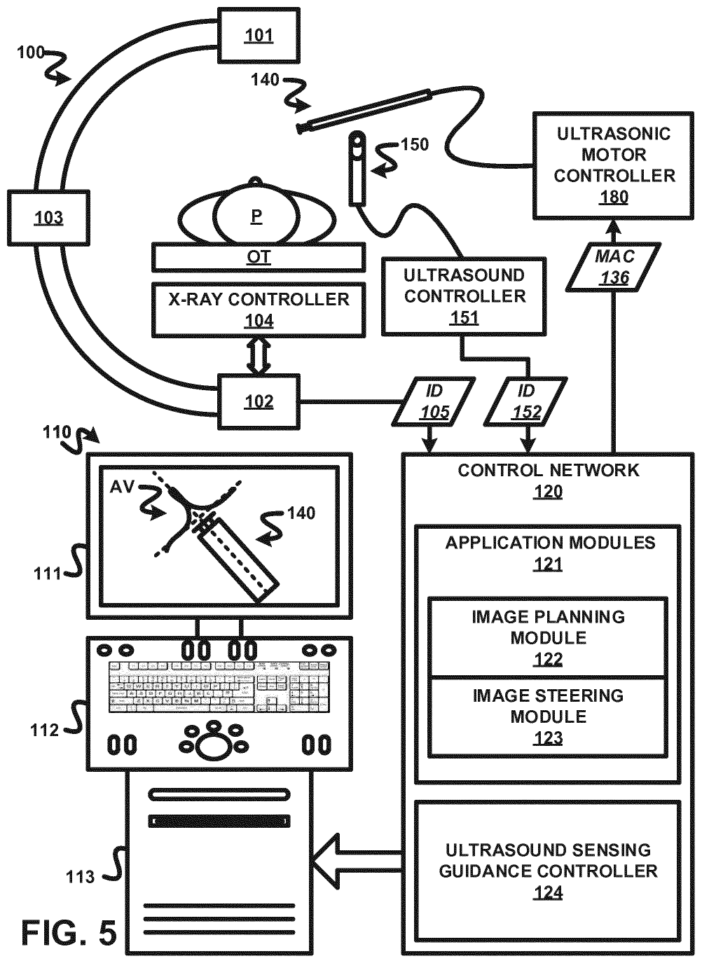

To facilitate a further understanding of the various inventions of the present disclosure, the following description of FIG. 5 teaches inventive principles of the present disclosure in the context of a steerable introducer 140 shown in FIG. 4. From this description, those having ordinary skill in the art will appreciate how to apply the inventive principles of the present disclosure for making and using additional embodiments of ultrasound sensing guiding systems of the present disclosure.

Referring to FIG. 4, steerable introducer 140 employs a shaft 141 and an end-effector 148.

A linear ultrasonic motor 143a and a motor controller 144a are housed within shaft 141 and motor 143a is rigidly coupled to shaft 141 via a rotary joint 146a. A rod 145a extends from shaft 141 and is rotatably coupled to end-effector 148 via a rotary joint 147a.

Similarly, a linear ultrasonic motor 143b and motor controller 144b are housed within shaft 141 and motor 143b is rigidly coupled to shaft 141 via a rotary joint 146b. A rod 145b extends from shaft 141 and is rotatably coupled to end-effector 148 via a rotary joint 147b.

Those having ordinary skill in the art of the present disclosure will appreciate that rods 145a and 145b may be jointly linearly actuated by respective motors 143a and 43b in a forward direction or a reverse direction to thereby translate end-effector 148 relative to shaft 141.

Those having ordinary skill in the art of the present disclosure will further appreciate that an exclusive linear actuation of rod 145a in a forward direction or a reverse direction pivots end-effector 148 relative to shaft 141. Similarly, an exclusive linear actuation of rod 145b in a forward direction or a reverse direction counter pivots end-effector 148 relative to shaft 141. Furthermore, any linear actuations of rod 145a and rod 145b in a forward direction or a reverse direction at different velocities will pivot end-effector 148 relative to shaft 141.

Those having ordinary skill in the art of the present disclosure will further appreciate that a linear actuation of rod 145a in a reverse direction and a linear actuation of rod 145b in a forward direction rotates end-effector 148 in a counter-clockwise direction relative to shaft 141. Conversely, a linear actuation of rod 145a in the forward direction and a linear actuation of rod 145b in the reverse direction rotates end-effector 148 in a clockwise relative to shaft 141.

Referring to FIG. 5, an ultrasound sensing guidance system of the present disclosure employs steerable introducer 140, an ultrasonic motor controller 180, a fluoroscopic imager 100 (e.g., a mobile c-arm as shown) and/or an ultrasound transducer 150, an ultrasound sensing guidance workstation 110 and a control network 110 for deploying an interventional tool within an anatomical object of a patient P lying prone on an operating table OT during a minimally invasive procedure of any type.

As known in the art, fluoroscopic imager 100 generally includes an X-ray generator 101, an image intensifier 102 and a collar 103 for rotating fluoroscopic imager 100. In operation as known in the art, an X-ray controller 104 controls a generation by fluoroscopic imager 100 of imaging data 105 illustrative of a fluoroscopic image of the anatomical object of patient P (e.g., a heart of patient P during a minimally invasive aortic valve replacement).

In practice, X-ray controller 104 may be installed within an X-ray imaging workstation (not shown), or alternatively installed within ultrasound sensing guidance workstation 110.

Ultrasound transducer 150 is any type of transducer suitable for a particular minimally invasive procedure (e.g., a Transesophageal echocardiography (TEE) transducer for a minimally invasive aortic valve replacement as shown). In operation as known in the art, an ultrasound controller 151 controls a generation by ultrasound transducer 150 of imaging data 152 illustrative of an ultrasound image of the anatomical object of patient P (e.g., a heart of patient P during a minimally invasive aortic valve replacement).

In practice, ultrasound controller 151 may be installed within an ultrasound imaging workstation (not shown), or alternatively installed within ultrasound sensing guidance workstation 110.

Workstation 110 may be assembled in a known arrangement of a standalone computing system employing a monitor 111, a keyboard 112 and a computer 112.

Control network 110 may be installed on computer 112, and employs application modules 121 including an image planning module 122 and an image steering module 123. Control network 110 may further includes an ultrasound sensing guidance controller 124.

Ultrasound sensing guidance controller 124 generally processes image data as known in the art for an illustration of the image on monitor 111. For example, ultrasound sensing guidance controller 124 may process X-ray image data 105 for an illustration of an X-ray image on monitor 111, and/or process ultrasound image data 152 for an illustration of an ultrasound image on monitor 111.

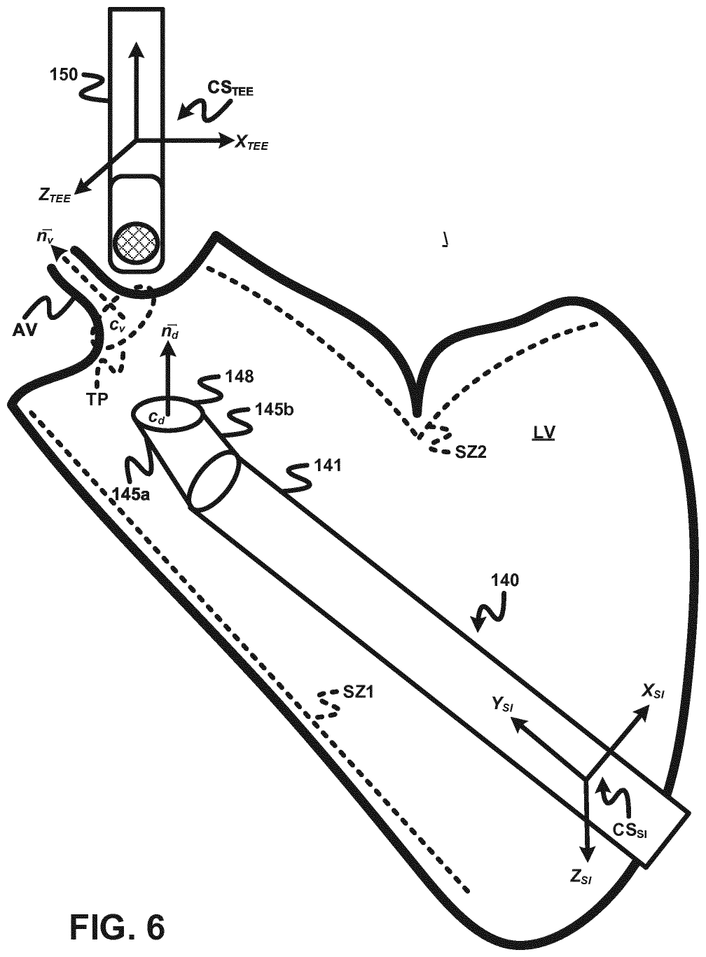

In support of the minimally invasive procedure, ultrasound sensing guidance controller 124 executes or accesses image planning module 122 to facilitate a user delineation of a coaxial alignment and/or a coplanar alignment of an interventional tool to a structure of anatomical object of patient P, such as, for example, an aortic valve AV of heart of patient P as shown in FIG. 6. To this end, ultrasound sensing guidance controller 124 controls an illustration of an X-ray image and/or an ultrasound image of the structure of the anatomical object on monitor 111, or concurrently or alternatively controls an illustration of a registered pre-operative image of the structure of the object on monitor 111 (e.g., a computed-tomography image or a magnetic resonance image). An operator of workstation 110 delineates, within the image(s), a target actuation position of an end-effector of steerable introducer 140 for achieving a coaxial alignment and/or a coplanar alignment of the interventional tool to the structure of anatomical object of patient P within the displayed image(s).

For example, the operator of workstation 110 may delineate, within the image(s), a target actuation position of an end-effector of steerable introducer 140 based on an intersection of valve annulus axis and valve annulus plane of a diseased aortic valve AV as shown in FIG. 6.

During the minimally invasive procedure, ultrasound sensing guidance controller 124 executes or accesses image steering module 123 to identify an end-effector of steerable introducer 140 within the displayed image(s) whereby ultrasound sensing guidance controller 124 may ascertain any necessary translational, pivot and/or rotation of the end-effector of steerable introducer 140 necessary to reach the target actuation position for achieving a coaxial alignment and/or a coplanar alignment of an interventional tool to the structure of the anatomical object of patient P.

For example, ultrasound sensing guidance controller 124 may identify, within the image(s), the end-effector of steerable introducer 140 relative to the delineated valve annulus axis and valve annulus plane of a diseased aortic valve AV as shown in FIG. 6 whereby ultrasound sensing guidance controller 124 ascertains any necessary translational, pivot and/or rotation of the end-effector of steerable introducer 140 necessary to reach the target actuation position for achieving the coaxial alignment with valve annulus axis and the coplanar alignment of valve annulus plane of the diseased aortic valve AV.

In practice, image steering module 123 may be built to implement the kinematics of steerable introducer 140. By implementing the kinematic model as known in the art of steerable introducer 140, an execution of image steering module 123 by ultrasound sensing guidance controller 124 enables ultrasound sensing guidance controller 124 to ascertain linear motion parameter(s) for linear actuator(s) of steerable introducer 140 to reach the target actuation position as will be further explained herein. Ultrasound sensing guidance controller 124 generates a motion actuation command 136 informative of desired linear motion parameter(s) for the linear actuator(s) and communicates motion actuation command 136 to ultrasonic motor controller 180 for actuating a translation, pivot and/or rotation by the linear actuator(s) of the end-effector of steerable introducer 140 to reach the target actuation position for achieving a coaxial alignment and/or a coplanar alignment of the interventional tool to the structure of the anatomical object of patient P. The generation of motion actuation command 136 involves a sensing by ultrasound sensing guidance controller 124 of an acoustic wave emitted by steerable introducer 140 as previously explained herein. The sensing of the acoustic wave facilitates a precise positioning of steerable introducer 140 to the target actuation position.

For example, ultrasound sensing guidance controller 124 may generate motion actuation command 136 to actuate a translational, pivot and/or rotation by the linear actuator(s) of the end-effector of steerable introducer 140 necessary to reach the target actuation position for achieving the coaxial alignment with valve annulus axis and the coplanar alignment of valve annulus plane of the diseased aortic valve AV as shown in FIG. 6. The generation of motion actuation command 136 involves a sensing by ultrasound sensing guidance controller 124 of an acoustic wave emitted by steerable introducer 140 that facilitates a precise positioning of steerable introducer 140 to the target actuation position for achieving the coaxial alignment with valve annulus axis and the coplanar alignment of valve annulus plane of the diseased aortic valve AV as shown in FIG. 6.

In practice, ultrasonic motor controller 180 may be a standalone controller or installed within ultrasound sensing guidance workstation 110.

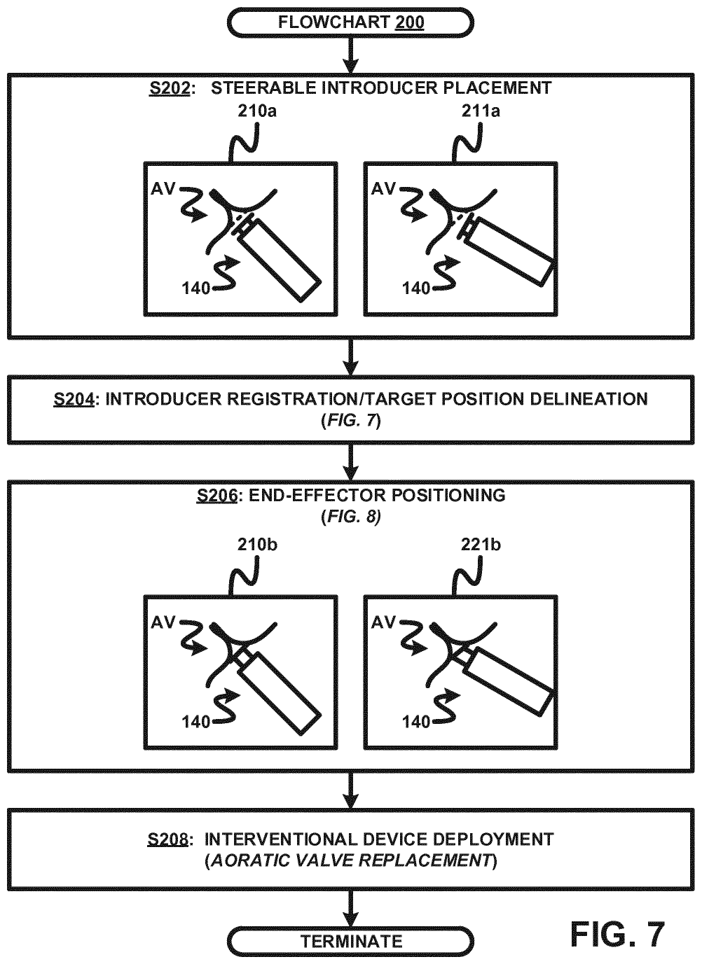

To facilitate a further understanding of the various inventions of the present disclosure, the following description of FIG. 7 teaches basic inventive principles associated with ultrasound sensing guidance methods of the present disclosure in the context of a minimally invasive aortic valve replacement. From this description, those having ordinary skill in the art will appreciate how to apply the inventive principles of the present disclosure for making and using additional embodiments of ultrasound sensing guidance methods of the present disclosure for any type of minimally invasive procedure suitable for a steerable introducer of the present disclosure.

Referring to FIG. 7, a stage S202 of a flowchart 200 encompasses a user placement of steerable introducer 140 (FIG. 5) into a heart of a patient as illustrated in a surgical image via fluoroscopic imager 100 (FIG. 5) encircling the thoracic cavity of the patient or via TEE probe 150 (FIG. 5) placed in the esophagus of the patient.

A transapical approach of stage S202 involves a small incision in a lower part of a chest, and a small puncture in left ventricle of the beating heart. The placement of steerable introducer 140 into the heart may position an end-effector of steerable introducer 140 anywhere within the left ventricle as illustrated in the surgical image via fluoroscopic imager 100 or TEE probe 150.

For example, a scenario 210a is an exemplary coaxial alignment and coplanar misalignment of an end-effector of steerable introducer 140 with an aortic valve AV of the heart.

By further example, a scenario 211a is an exemplary coaxial misalignment and coplanar misalignment of an end-effector of steerable introducer 140 with an aortic valve AV of the heart.

A transaortic approach of stage S202 involves a small incision in an upper part of a chest of the patient, and a small puncture in the aorta of the beating heart of the patient. The placement of steerable introducer 140 into the heart may position (i.e., location and orientation) an end-effector of steerable introducer 140 anywhere within the aorta as illustrated in the surgical image via TEE probe 150 or alternatively fluoroscopic imager 100.

Those having skill in the art will appreciate exemplary transapical scenarios of the transaortic approach analogous to the scenarios 210a and 211a A stage S204 of flowchart 200 encompasses ultrasound sensing guidance controller 124 (FIG. 5) facilitating a registration of steerable introducer 140 to the applicable imaging modality, fluoroscopic imager 100 or TEE probe 150.

In practice, the registration may be executed by any known technique in the art for generating a transformation matrix between an actuation coordinate system of steerable introducer 140 to an image coordinate system of the applicable imaging modality.

The actuation coordinate system of steerable introducer 140 defines a reference point for tracking a position of the end-effector of steerable introducer 140 within the actuation coordinate system, particularly in terms of a location of specified point of the end-effector (e.g., a central point) and the orientation of the end-effector about the location of the specified point of the end-effector.

The image coordinate system of the applicable imaging modality defines a reference point for identifying positions of anatomical structures and of steerable introducer 140 within the live images of the anatomical object.

Also in practice, the actuation coordinate system of steerable introducer 140 is assumed to be static in view of a shaft of steerable introducer 140 being anchored in a heart muscle. By comparison, the image coordinate system may be static in view of a fixed positioning of the applicable imaging modality whereby the initial registration is maintained over an execution of flowchart 200. Conversely, the image coordinate system may be dynamic in view of a changing positioning of the applicable imaging modality whereby the initial registration is updated as needed over an execution of flowchart 200.

Stage S204 of flowchart 200 further encompasses ultrasound sensing guidance controller 124 facilitating a surgeon delineation or an image delineation of a target position of the end-effector of steerable introducer 140 within the live image of the anatomical object.

In one embodiment, a surgeon may outline a desired target position of the end-effector of steerable introducer 140 within the live image of the anatomical object.

In a second embodiment, ultrasound sensing guidance controller 124 performs an automatic segmentation of the targeted structure within the live image of the anatomical object as known in the art, and determines a desired target position of the end-effector of steerable introducer 140 relative to the segmented structure.

In practice, the delineated target position may be described as a plane defined by a center and a unit vector normal to the plane.

A stage S206 of flowchart 200 encompasses an actuation of steerable introducer 140 by ultrasound sensing guidance controller 124 for steering the end-effector thereof to the delineated target position for achieving a coaxial alignment and/or a coplanar alignment of the end-effector of steerable introducer 140 with an aortic valve AV of the heart as shown in live images of the heart (e.g., X-ray or ultrasound).

For example, a scenario 210b is an exemplary translation motion of the end-effector to thereby achieve a coaxial alignment and a coplanar alignment of an end-effector of a steerable introducer 140 with an aortic valve AV of the heart.

By further example, a scenario 211b is an exemplary translation motion and pitch motion of the end-effector to thereby achieve a coaxial alignment and a coplanar alignment of an end-effector of steerable introducer 140 with aortic valve AV of the heart.

Those having skill in the art will appreciate exemplary scenarios of the transaortic approach analogous to the transapical scenarios 210b-152b.

A stage S208 of flowchart 200 encompasses a deployment of an artificial valve by passing a balloon catheter supporting the artificial valve through steerable introducer 140 and the end-effector thereof guiding a positioning of a balloon catheter supporting an artificial valve. Alternatively, the balloon catheter may be securely or separably adjoined to the end-effector of steerable introducer 140 during stages S202 and S204 whereby ultrasound sensing guidance controller 124 via the live image identifies and accounts for the balloon catheter during the placement of steerable introducer 140 of stage S202 and the positioning of the end-effector during stage S206.

Flowchart chart 200 is terminated upon deployment of the artificial valve.

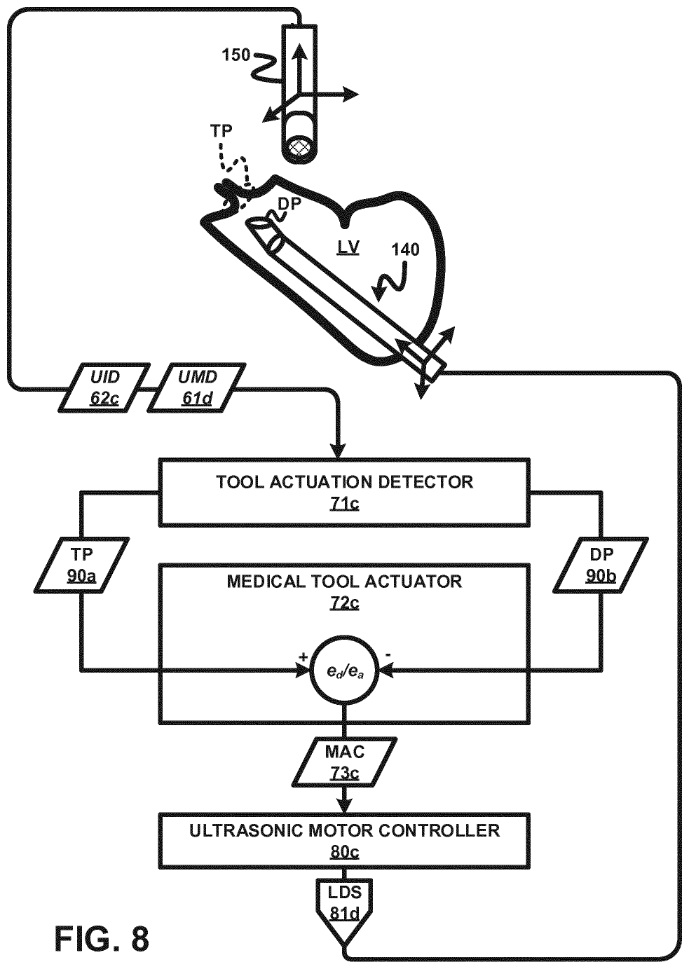

FIG. 8 illustrate a control loop of executed during stage S206 (FIG. 7). Referring to FIG. 8, the control loop includes an TEE probe 150 generating ultrasound image data 62c by an emission and a reception of ultrasound waves symbolized by the bi-directional waves within anatomical region 10 whereby an ultrasound image illustrative of a positioning of the end-effector of steerable introducer 140 within anatomical region 10 is utilized to delineate a target actuation position 90a of the end-effector of steerable introducer 140 within left ventricle LV of beating heart and to detect a current actuation position 91a of the end-effector of steerable introducer 140 within left ventricle LV of beating heart.

As shown in FIG. 8, the control loop further includes a medical tool actuator 72c of an ultrasound sensing guidance controller of the present disclosure ascertaining a position distance error signal e_{d} as a dot differential between the center cᵥ of target actuation position 90a and the center c_{d} of detected actuation position 91a in accordance with the previously described equation [1].

Medical tool actuator 72c may further ascertain an alignment error signal eₐ as a dot product of two planes normal indicative of an angular error in accordance with the previously described equation [2].

Medical tool actuator 72c may further ascertain a safety error signal eₛ (not shown) as a dot differential between the center cᵥ of target actuation position 90a and a spot location cₛ of an anatomical structure within anatomical region 10 closest detected actuation position 91a in accordance with the previously described equation [3].

From the position distance error signal and alignment error signal (if applicable), medical tool actuator 72c generates motion actuation command 73c for minimizing the error signal (e.g. driving the error signal(s) to zero within the context of safety error signal (if applicable)). Medical tool actuator 72c communicates motion actuation command 73c whereby ultrasound motor controller 80c processes the applicable error signal(s) as inputs to an inverse kinematics computation of the end-effector of steerable introducer 140 to ascertain a linear drive signal 81d for ultrasonic motor of steerable introducer 140 to thereby actuate the necessary translation of the end-effector of steerable introducer 140 to target position 90a.

As ultrasound motor of steerable introducer 140 translates the end-effector of steerable introducer 140 to target position 90a, ultrasound transducer 50a generates acoustic sensing data 61a informative of a sensing of an acoustic wave emitted ultrasonic motor of steerable introducer 140 whereby a motion actuation detector 71c of the ultrasound sensing guidance controller generates an acoustic wave image of a waveform of the acoustic wave. By detecting the peaks of waveform of the acoustic wave, motor actuation detector 71c ascertains the detected actuation position 91b of the end-effector of steerable introducer 140 whereby medical tool actuator 72c continually generates the error signal(s) until the end-effector of steerable introducer 140 reaches target actuation position 90a.

In practice, the detected actuation position 91b may be detected based on both an ultrasound image of the left ventricle LV of the beating heart and the acoustic wave image whereby one of the images serves as the primary source of detected actuation position 91b and the other image serves to confirm detected actuation position.

Also in practice, application modules 74-77 (FIG. 1) or equivalents thereof may be incorporated in support of the control loop.

Referring to FIGS. 1-8, those having ordinary skill in the art will appreciate numerous benefits of the present disclosure including, but not limited to, an improvement over ultrasound guidance systems and methods by the inventions of the present disclosure in providing control of a medical tool using combined ultrasound imaging and sensing for accurate positioning of the medical tool by a novel and unique utilization of an ultrasonic motor to produce acoustic waves detectable for precise positioning of the medical tool.

Furthermore, as one having ordinary skill in the art will appreciate in view of the teachings provided herein, features, elements, components, etc. described in the present disclosure/specification and/or depicted in the Figures may be implemented in various combinations of electronic components/circuitry, hardware, executable software and executable firmware and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk-read only memory (CD-ROM), compact disk-read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive ultrasound sensing guidance systems and methods, (which embodiments are intended to be illustrative and not limiting), it is noted that modifications and variations can be made by persons having ordinary skill in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. An ultrasound sensing guidance system, comprising:
a medical tool including an ultrasonic motor structurally configured to actuate the medical tool relative to an anatomical region;
an ultrasound transducer structurally configured to generate acoustic sensing data indicative of a sensing by the ultrasound transducer of an acoustic wave emitted by the ultrasonic motor as the ultrasonic motor actuates the medical tool relative to the anatomical region; and
an ultrasound sensing guidance controller structurally configured to control an actuation of the medical tool by the ultrasonic motor responsive to a generation of the acoustic sensing data by the ultrasound transducer.

2. The ultrasound sensing guidance system of claim 1, wherein the ultrasound sensing guidance controller includes:
a tool actuation detector structurally configured to detect the actuation of the medical tool by the ultrasonic motor responsive to a generation by the ultrasound transducer of acoustic sensing data; and
a medical tool actuator structurally configured to control the actuation of the medical tool responsive to a detection by the tool actuation detector of the actuation position of the medical tool relative to the anatomical region.

3. The ultrasound sensing guidance system of claim 2,
wherein the tool actuation detector detects an actuation position of the medical tool relative to the anatomical region;
wherein the medical tool actuator generates a motion actuation command instructive of an actuation of the medical device from the actuation position to a target position of the medical tool relative to the anatomical region; and
wherein the ultrasonic motor actuates the medical tool from the actuation position to the target position responsive to a generation of the motion actuation command by the medical tool actuator.

4. The ultrasound sensing guidance system of claim 3,
wherein the medical tool actuator derives the motion actuation command from an error differential between the actuation position and the target position of the medical tool relative to the anatomical region.

5. The ultrasound sensing guidance system of claim 1, wherein the ultrasound sensing guidance controller includes:
a motion command analyzer structurally configured to ascertain a status of the control by the ultrasound sensing guidance controller of the actuation of the medical tool by the ultrasonic motor derived from a waveform analysis of the acoustic wave by the motion command analyzer.

6. The ultrasound sensing guidance system of claim 1, wherein the ultrasound sensing guidance controller includes:
a control delay compensator structurally configured to detect any time delay in the control by the ultrasound sensing guidance controller of the actuation of the medical tool by the ultrasonic motor.

7. The ultrasound sensing guidance system of claim 1, wherein the ultrasound sensing guidance controller includes:
a motion condition analyzer structurally configured to determine an actuation condition of the ultrasonic motor derived from an analysis by the motion condition analyzer of any difference between an expected waveform of the acoustic wave and an actual received waveform of acoustic the acoustic wave.

8. The ultrasound sensing guidance system of claim 1, wherein the ultrasound sensing guidance controller further includes:
a diagnostic manager structurally configured to diagnosis any problem of the ultrasonic motor indicated by a waveform of the acoustic wave.

9. The ultrasound sensing guidance system of claim 1,
wherein the ultrasound transducer is further structurally configured to generate ultrasound imaging data indicative of an ultrasound imaging of the medical tool relative to the anatomical region as the ultrasonic motor actuates the medical tool to the target actuation position relative to the anatomical region; and
wherein the ultrasound sensing guidance controller is further structurally configured to a delineation of a target position within the imaging of the medical tool relative to anatomical region responsive to a generation of the ultrasound imaging data by the ultrasound transducer.

10. An ultrasound sensing guidance controller for an ultrasound transducer and a medical tool including an ultrasonic motor, the ultrasound sensing guidance controller comprising:
a tool actuation detector structurally configured to detect an actuation of the medical tool relative to an anatomical region responsive to a generation by the ultrasound transducer of acoustic sensing data indicative of a sensing by the ultrasound transducer of an emission of an acoustic wave by the ultrasonic motor as the ultrasonic motor actuates the medical tool relative to the anatomical region; and
a medical tool actuator structurally configured to control the actuation of the medical tool by the ultrasonic motor relative to the anatomical region responsive to a detection by the tool actuation detector of the actuation of the medical tool by the ultrasonic motor relative to the anatomical region.

11. The ultrasound sensing guidance controller of claim 10,
wherein the tool actuation detector detects an actuation position of the medical tool relative to the anatomical region; and
wherein the medical tool actuator generates a motion actuation command instructive of an actuation of the medical tool from the sensed position to a target position of the medical tool relative to the anatomical region.

12. The ultrasound sensing guidance controller of claim 10, further comprising:
a motion command analyzer structurally configured to ascertain a status of the control by the medical tool actuator of the actuation of the medical tool by the ultrasonic motor derived from a waveform analysis of the acoustic wave by the motion command analyzer.

13. The ultrasound sensing guidance controller of claim 10, further comprising:
a control delay compensator structurally configured to detect any time delay in the control by the medical tool actuator of the actuation of the medical tool by the ultrasonic motor.

14. The ultrasound sensing guidance controller of claim 10, further comprising:
a motion condition analyzer structurally configured to determine an actuation condition of the ultrasonic motor derived from an analysis by the motion condition analyzer of any difference between an expected waveform of the acoustic wave and an actual received waveform of acoustic the acoustic wave.

15. The ultrasound sensing guidance controller of claim 10, further comprising:
a diagnostic manager structurally configured to diagnosis any problem of the ultrasonic motor indicated by a waveform of the acoustic wave.

16. An ultrasound sensing guidance method for an ultrasound transducer and a medical tool including an ultrasonic motor, the ultrasound sensing guidance method comprising:
an ultrasound sensing guidance controller controlling an actuation of the medical tool by the ultrasonic motor relative to an anatomical region; and
the ultrasound transducer generating acoustic sensing data indicative of a sensing by the ultrasound transducer of an emission of an acoustic wave by the ultrasonic motor as the ultrasonic motor actuates the medical tool relative to the anatomical region,
wherein the ultrasound sensing guidance controller controls the actuation of the medical tool by the ultrasonic motor relative to the anatomical region responsive to the generation of the acoustic sensing data by the ultrasound transducer.

17. The ultrasound sensing guidance method of claim 16,
wherein the ultrasound sensing guidance controller controlling the actuation of the medical tool by the ultrasonic motor relative to an anatomical region includes:
the ultrasound sensing guidance controller detecting an actuation position of the medical tool relative to the anatomical region;
the ultrasound sensing guidance controller generating a motion actuation command instructive of an actuation of the medical tool from the sensed position to a target position of the medical tool relative to the anatomical region; and
the ultrasonic motor actuating the medical tool from the sensed position to a target position in response to the motion actuation command.

18. The ultrasound sensing guidance method of claim 17,
wherein the ultrasound sensing guidance controller derives the motion actuation command from an error differential between the actuation position and the target position of the medical tool relative to the anatomical region.

19. The ultrasound sensing guidance method of claim 16, wherein the ultrasound sensing guidance controller controlling the actuation of the medical tool by the ultrasonic motor relative to an anatomical region includes at least one of:
the ultrasound sensing guidance controller ascertaining a status of the control by the medical tool actuator of the actuation of the medical tool by the ultrasonic motor derived from a waveform analysis of the acoustic wave by the motion command analyzer;
the ultrasound sensing guidance controller detecting any time delay in the control by the medical tool actuator of the actuation of the medical tool by the ultrasonic motor;
the ultrasound sensing guidance controller determining an actuation condition of the ultrasonic motor derived from an analysis by the motion analyzer of any difference between an expected waveform of the acoustic wave and an actual received waveform of acoustic the acoustic wave; and
the ultrasound sensing guidance controller diagnosing any problem of the ultrasonic motor indicated by a waveform of the acoustic wave.

20. The ultrasound sensing guidance method of claim 17, further comprising:
the ultrasound transducer generating ultrasound imaging data indicative of an ultrasound imaging of the medical tool relative to the anatomical region as the ultrasonic motor actuates the medical tool to the target actuation position relative to the anatomical region; and
the ultrasound sensing guidance controller controlling a delineation of the target position within the imaging of the medical tool relative to anatomical region responsive to a generation of the ultrasound imaging data by the ultrasound transducer.
